(19) United States
(12) Patent Application Publication
(10) Pub. No.: US 2007/0060508 A1
(43) Pub. Date: Mar. 15, 2007

(54) Binding molecules

(76) Inventors: Udo Haberl, Baesweiler (DE); Christian Frosch, Karlsruhe (DE); Hans-Georg Frank, Baesweiler (DE); Andreas Rybka, Herten (DE); Raimund Wieser, Aachen (DE)

Correspondence Address: JACOBSON HOLMAN PLLC, 400 SEVENTH STREET N.W., SUITE 600, WASHINGTON, DC, 20004, US

(21) Appl. No.: 11/389,667

(22) Filed: Mar. 27, 2006

Related U.S. Application Data
(63) Continuation of application No. 10/523,716, now abandoned.
(60) Provisional application No. 60/401,032, filed on Aug. 6, 2002.

(30) Foreign Application Priority Data
Aug. 6, 2002 (EP) 02017525.3
May 15, 2003 (EP) 03010892.2

(51) Int. Cl. A61K 38/12; C07K 7/64; C07K 7/60

(52) U.S. Cl. 514/9; 530/317

(57) ABSTRACT

The invention pertains to binding molecules consisting of a carrier structure of at least one cyclic molecular subunit and at least two side chain subunits, wherein the side chain subunits are polypeptide chains consisting of natural and/or unnatural D- and/or L-amino acids and the side chain subunits are covalently bonded to the support structure.

In numerous diseases a change of the active concentration of antibodies, enzymes and endocrine, paracrine or autocrine messengers occur within the scope of the pathogenetic process. Such biologically active binding molecules may be of the protein family (examples: insulin, growth factors, cytokines, releasing hormones, antibodies, enzymes, etc.), the lipid class (prostaglandins and similar fatty acid derivatives), the steroid class (examples: glucocorticoids, mineral corticoids). Hereinafter, the term cytokine is to be interpreted in a prototypical manner for biologically active binding molecules involved in binding processes or signal transducing processes. Cytokines are biological messengers with high specificity and defined activity. They generate their biological activity by binding to cytokine-specific receptors which are preferably provided on the surface of target cells. When the messenger contacts the receptor, in the typical case the latter will trigger an intracellular signal transducing cascade resulting in the biological effect within the cell or tissue. Typical effects which may be triggered by such receptors are, e.g., the entry of a cell into the cell cycle and therewith an increase of the multiplication rate or also the triggering of apoptosis, a cell death form. Also in inflammatory processes, in the immunologic system and many local regulating processes of the mammalian body cytokines play an important role in the homoeostasis of the respective organism. With many cytokines, such as, e.g., most members of the interleukine cytokine family, the receptor at the cell surface consists of different proteins which converge due to the binding of the cytokine. Hence, many cytokines have specific binding sites which have to bind to two or more receptor proteins at the same time to trigger the effect with high efficiency. This indicates that in the cytokine receptor interaction precisely defined distances and spatial orientations of the various binding regions in the cytokine molecule with respect to each other are critical and necessary for the complete display of the effect. Moreover, with some cytokines the cytokine as such consist of different proteins which are required to combine in the course of the receptor interaction (C. Aul, W. Schneider (Eds.), Biological Activities and Clinical Efficacies, 1997, Springer Verlag Berlin). Recently, a number of cytokines has recombinantly been prepared to use them as pharmaceuticals. Indications in which such pharmaceuticals have already been used are, e.g., severe, life-threatening tumor diseases and immunodeficiency diseases.

However, the recombinant preparation has several drawbacks:

1. The preparation of recombinant proteins—for example cytokines—requires a high expenditure during the “set-up”. It has to be ensured in a complicated manner that the cells used for expression exactly produce the protein in the exact “native” conformation. A multiplicity of tests is required to find a suitable expression system with cells and a vector and conditions for a high expression and stability in solution. Often the “set-up” conditions are not transferable to a large-scale fermentation. Even if optimal conditions are found, the expression systems are often found to be labile and trouble-prone.

2. Since the cytokines are to be administered regularly, it has to be ensured that they are purified after expression which regularly is performed in bacteria and subsequently are absolutely free from bacterial contaminants to avoid a systemic allergy. This necessitates the use of complex methods which have an enormous impact on the pricing.

3. Many messengers such as, e.g., cytokines, do not have only one binding site for the receptor but there exist further domains which are in most cases not completely understood and which may possibly induce signal paths other than the envisaged ones. This might also explain, i.a., the frequently occurring side effects which partially make a treatment with these substances impossible.

4. Proteins prepared by recombination often have tertiary structures deviating from those of native proteins and therefore are recognized as “alien” by the human body. The antibodies induced thereby neutralize the cytokine and result in a loss of activity of the cytokine.

5. Recombinant proteins often exhibit a remarkable lability to proteolytic—i.e. protein degrading—enzymes. This necessitates a frequent administration in relatively high concentrations which on the one hand stresses the patient and on the other hand makes the therapy very costly.

An economic alternative to the recombinant preparation of a cytokine are organic-chemically accessible, completely synthetic mimetics of biologically active proteins such as, e.g., cytokines.

An approach taken here is, e.g., the artificial design of catalytically active centers of known proteins, although research is still in its infancy and up to the present only stabilizations of peptide structures (β sheet, helices, turns) and in some cases purposive complexations of smaller molecules could be realized.

Another approach is the computer-assisted design of small, specific binding molecules of, e.g., peptide nature. Such small specific binding molecules usually have a sequence of less than 100 amino acids. Due to the size thereof, such molecules cannot contribute to the association of receptor subunits which would be required for a complete cytokine activity. Normally, they will also not be designed to be highly affine for two or more binding sites on a large receptor molecule at the same time.

By now, peptides can chemically be synthesized by numerous standardized methods quite easily, although an individual synthetic strategy has to be developed for each peptide. Normally, the peptides are synthesized on solid phases with the operations of activating an N-terminally protected amino acid, coupling, washing, deblocking, activating being repeated until the desired peptide is finished. Said product is removed from the solid phase, purified by HPLC and subsequently transferred to further investigations such as, e.g., sequence verification and biological tests. Generally, the synthesis is the simpler and the more reliable the shorter the peptide is.

Some few investigations of peptide oligomers demonstrated that chemically synthesized and oligomerized peptides show a biological activity, said activity regularly being below that of the native protein, like, for instance, in the case of erythropoetine (patent no. WO 96/40772), but often stronger than the effect of the monomer.

Up to the present, the preparation of oligomers has also been performed using mostly commercially available “cross-linkers” according to standardized methods described in the literature. Frequently, these molecular structures, which are not found in the body, are biologically inert and “inconspicuous” with the intention that the special chemical composition does not produce immunological reactions.

Peptides have an extremely short in vivo half life, i.e., they are degraded and excreted very fast by endogenous enzymes. Various approaches are applied to metabolically stabilize peptides and proteins. On the one hand, unnatural amino acids which cannot be cleaved are used in synthesis, on the other hand proteins are modified by inert chemical groups to protect them from degradation. A known example for this is the PEG intron (Schering-Plough), an interferon modified with polyethylene glycol.

Depending on the planned geometrical arrangement of the peptides as specific binding molecules, one has to use the most different strategies for the preparation of possible support structures and to develop an individual support structure for each special problem. Up to the present, suitable approaches to solve this problem in a synthetical manner by developing a universally applicable strategy wherein both the number and the type and the mutual orientation of the specific binding molecules are flexible do not exist. The previously used approaches for preparing suitable support structures are based either on the formation of disulfide bridges ((a) Nomiz, M., Utani, A., Shiraishi, N., Yamada, Y., Roller, P.; “Synthesis and conformation of the trimeric coiled-coil segment of laminin”; Int J Pept Protein Res (40(1); 1992; 72-79) or the use of dendrimer-like structures on the basis of lysine ((a) Carrithers, M. D., Lerner, M. R.; Synthesis and characterization of bivalent peptide ligands targeted to G-protein-coupled receptors”; Chem Biol 3(7); 1996; 537-542; (b) Wada, T., Okada, K., Nakamori, M., Mochizuki, E., Inaki, Y.; “Synthesis and properties of oligolysine and oligoglutamic acid derivatives containing nucleosides”; Nucleic Acids Symp Ser 29; 1993; 79-80; (c) Henkel, W., Vogl, T., Echner, H., Voelter, W., Urbanke, C., Schleuder, D., Rauterberg, J.; “Synthesis and folding of native collagen III model peptides”; Biochemistry 38(41); 1999; 13610-22) and thus the approaches as such constitute a limitation of the possibilities.

It is the object of the present invention to provide binding molecules with a high physiological activity which do not have the above-mentioned drawbacks of binding molecules of the state of the art.

Surprisingly, the object forming the basis of the invention is attained by a binding molecule consisting of a support structure of at least one cyclic molecular subunit and at least two side chain subunits, wherein the side chain subunits are polypeptide chains synthesized from natural and/or unnatural D- and/or L-amino acids and wherein the side chain subunits are covalently bonded to the support structure.

The cyclic molecular subunit is preferably a cyclic polypeptide synthesized from natural or unnatural D- and/or L-amino acids, an aromatic ring, an aromatic ring system, a polylactone, a polylactam, benzenetriamide, trihydroxybenzoic acid, a trilactone or a trilactam. In some preferred embodiments with exactly two side chains to be linked the cyclic support structure can also be structurally minimized and be replaced by a linear symmetrical structure.

The amino acids forming the cyclic polypeptide are advantageously linked by peptide bonds. A cyclic molecular subunit within the meaning of the invention is not present if a cyclic structure is formed by the formation of a disulfide bridge. This type of a cyclic peptide structure is a constituent part of many naturally occurring proteins. Due to the lability thereof, it is not suited to be used as the central cyclic molecular subunit of the support structure within the meaning of the invention. Of course, this does not exclude that the binding molecules of the invention also contain disulfide bridges in addition to a more stabile cyclic molecular subunit of the support structure. Such additional disulfide bonds will preferably be located at sites relatively distant from the support structure and will be introduced to stabilize a specific sterical configuration within and/or between two or more side chains.

If the cyclic molecular subunit is a cyclic polypeptide, in preferred embodiments said at least two side chain subunits of the binding molecule are bonded to the cyclic molecular subunit by amino acid side chain moieties of the amino acids lysine, serine, threonine, glutamate, asparagine and/or aspartate of the cyclic molecular subunit.

The cyclic polypeptide preferably consists of 2 to 10, in particular of 2, 3, 4 or 6 amino acids.

In preferred embodiments, the cyclic molecular subunit has one of the structures

a) benzenetriamide:

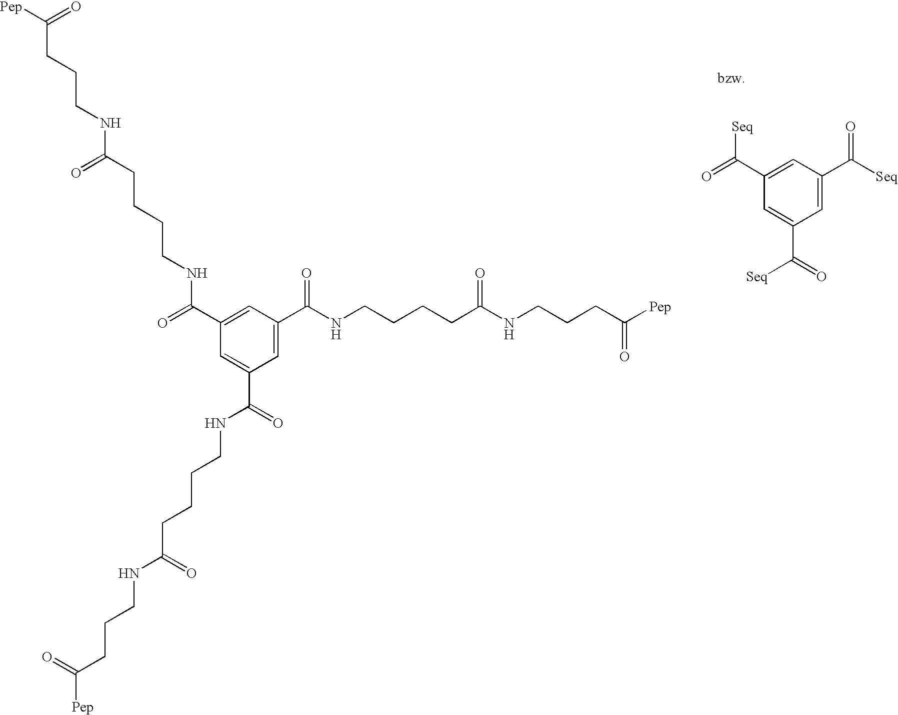

b) cyclohexapeptide:

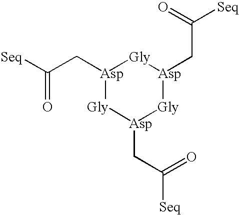

or a cyclohexapeptide of the sequence DKDKDK with one up to three side chain peptides Seq,

c) trilactone:

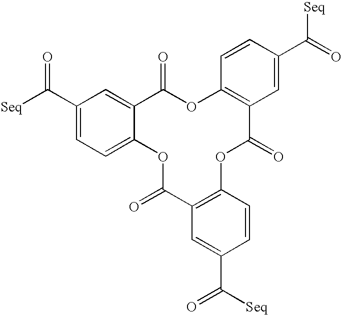

d) trilactame:

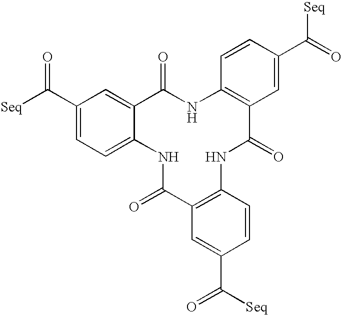

e) trihydroxybenzoic acid support structure:

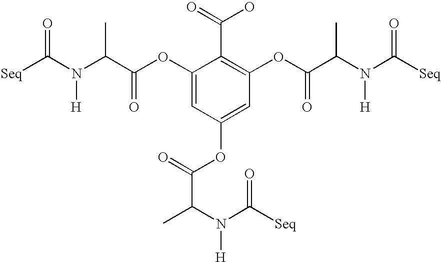

f) cyclic dipeptide: cyclo-L-Ala-L-Lys, derivatized with Seq,

wherein Seq or Pep represent the side chain subunits or —OH. According to the invention, the molecules comprise at least two side chain subunits which may be identical or also different from each other.

If said at least two side chain subunits of the binding molecule of the invention are polypeptide chains, they may have the same amino acid sequence or different sequences.

The polypeptide chains of the side chain subunits preferably consist of 5 to 60, preferably 10 to 50, in particular 12 to 40 amino acids. The molecular weight of such polypeptide chains is preferably not greater than 10 kDa, in particular below 8 kDa or below 5 kDa.

It is considered part of the present invention that the sterical position of two or more side chains on a cyclic support structure or at the ends of a linear connecting molecule can be fixed during the synthesis of the whole molecule by introduction of disulfide bonds between two sterically suitably neighbouring residues (e.g. cystein or homocystein) of the respective side chains. For this purpose, the amino acid sequences of the side chains can be modified or optimized to contain one or more suitable SH-containing residues at suitable location.

Another object of the present invention are binding molecules wherein the side chain subunits are oligo or polynucleic acids consisting of natural and unnatural nucleic acid units. Then, the nucleotides may be connected in a natural manner or they may exist as peptide nucleic acids (PNA). The molecular weight of such side chain subunits is preferably not greater than 10 kDa, in particular below 8 kDa or below 5 kDa. For example, such binding molecules of the invention may be used to bind to DNA-binding proteins or protein complexes with several DNA-binding sites.

In a preferred embodiment the side chain subunits are connected with the support structure by spacer molecular subunits (spacers) especially consisting of natural and unnatural carbohydrates or nucleic acids.

Preferably, the free ends of the side chain subunits are covalently bonded to suitable protective groups to prevent exopeptidase degradation. Depending on the type of bonding between the side chain subunits and the support structure, the N- or the C-terminus may be the free ends. Preferred protective groups are D-amino acids, preferably as di- or tripeptide. Furthermore, protective groups usually employed within the scope of solid phase synthesis and monomeric and polymeric carbohydrates may be used.

To prevent endopeptidases from cleaving the side chain peptides, the internal amino acids may also covalently be bonded to protective groups. Preferred protective groups are carbohydrates which preferably are selected from glucose, mannose, N-acetyl glucosamine or N-acetylgalactosamine or sialic acid.

In preferred embodiments the unnatural amino acids which may be a constituent part of the binding molecule of the invention have the formula NH₂(CH₂)ₙCOOH with n=2 to 8, in particular NH₂(CH₂)₂COOH, NH₂(CH₂)₃COOH or NH₂(CH₂)₄COOH.

The present invention enables the development and preparation of fully synthetical mimetics of physiologic binding molecules which overcome the above-mentioned drawbacks of the state of the art. The present invention designs support structures on the skeleton of which binding molecules are oriented such that the binding properties of a biologically active binding molecule are mimiced. A great advantage of the invention is the simple synthetic preparation of the binding molecules of the invention. The organic-chemical synthesis can be carried out cheaper and controlled better than the recombinant preparation of an analogous protein molecule. Moreover, in principle also unnatural amino acids, e.g., D-amino acids or other suitable monomeric units can be used of which a better compatibility or immunologic tolerance is to be expected.

Inter alia, the invention described here overcomes the drawbacks of the state of the art in the following items:

a) It describes an easily preparable cyclic support structure which usually is easily preparable and available and which can be used for the positioning of specific binding molecules.

b) The cyclic structure of the support structure enables the positioning of specific binding molecules in practically any desired spatial orienting by varying the ring size and the distances between the sites the side chain subunits are bonded to and optionally by incorporating suitable spacer molecules. Hence, the binding molecules of the invention distinctly differ from those according to the state of the art where only a random crosslinking of peptides with synthetic molecules was performed and wherein a defined spatial orientation of the molecule components is not possible.

c) By selecting suitable monomeric units, the cyclic support structure can be synthesized on the solid phase in a defined sequence and the specific binding molecules can be attached or synthesized as side chain subunits. In the case of amino acids as monomeric units, this enables the complete solid phase synthesis of peptides oriented in a parallel or antiparallel manner on the support structure.

d) The support structure is flexible with respect to the number, type and length of the specific binding molecules to be attached as side chain subunits.

e) According to the invention, amino acid sequences oriented in a parallel or antiparallel manner can be coupled with the support structure in one synthesis step in the solid phase and optionally synthesized therewith in one operation. The latter is advantageous for the yield and purity of the product and the production costs. Here, a parallel orientation refers to the side chain subunits all being bonded to the support structure by the N- or C-terminal end in the same direction. An antiparallel orientation refers to at least one side chain subunit being bonded N-terminally and at least one being bonded C-terminally to the support structure.

The flexibility with the planned positioning of specific binding molecules may be increased by using unnatural amino acids and D-amino acids. The use of unnatural and D-amino acids or lactones and lactams increases the stability against proteolytic degradation. Additional functionalities increasing stability and half life can be introduced due to the variability of the support structures. This applies to, e.g., glycosylations, acetylations, disulfide-bridging or other chemical derivatizations known to the skilled person from protective group chemistry.

If the specific binding molecules to be positioned on the support structure of the invention are peptides, they are orientable and synthesizable in a parallel and antiparallel manner on the support skeleton.

A preferred embodiment of the support structures of the invention are cyclic peptides. Suitable state-of-the-art protective group strategies are available for cyclic peptides. Cyclic peptides degrade slower in vivo since they can only be cleaved by endopeptidases. Moreover, according to the process of the invention they can be synthesized with an increased proteolysis resistance by incorporating unnatural amino acids such as, e.g., D-amino acids. In case of exactly two functional side chains to be linked the support structure can be minimized to a linear structure.

Interleukin-2 Receptor-Specific Binding Molecules

In one embodiment of the invention there are provided synthetic binding molecules which—in analogy to interleukin-2 (IL2)—have the capability to bind to IL2 specific receptors with high affinity. IL2 is a cytokine used in tumor therapy. It is a protein consisting of 133 amino acids and having a molecular weight of 15.4 kDa. IL2 binds to specific receptors (IL2R) whereby the IL2-specific intracellular signals are triggered. The highly amine receptor is a receptor complex consisting of the subunits a, b and g (also designated as p55, p75 and p64, resp.). Stimulating the receptor complex consisting of p75 and p64 is sufficient to induce the complete activity of IL2.

IL2 has stimulating effects on the growth of T and B lymphocytes, activates cytotoxic and cytolytic NK cells. Thus it has a central significance in the regulation of the immune response. Thus, IL2 is of fundamental importance in the immune response to tumors and inflammatory reactions. One of the mechanisms which is of importance for the tumor defense with IL2 seems to be the induction of LAKs (“lymphokine activated killer cells”). These cells are able to destroy tumor cells.

In recent years various attempts in tumor therapy with IL2 have been made. By now a recombinantly prepared product (Aldesleukin, Proleukin by Chiron) for the therapy of metastasizing renal cell carcinoma and metastatic myeloma is marketed. The recombinant preparation has high development, approval and production costs which results in correspondingly high market prices. Moreover, recombinant production processes are trouble-prone to a high degree. Only about 30% of the treated patients show a reaction to the IL2 therapy. A broad clinical application of IL2 is prevented by the extremely short half life within the human organism (below 10 min) and a broad toxicity, the treatment-related mortality rate observed during a study being 4%.

In addition to nausea, vomiting and diarrhoea, above all the IL2 therapy is accompanied by organ dysfunctions and neuropsychiatric effects. The toxicity is so high that only 10% of the dose determined to be optimal in animal studies is used with humans. Thus, the therapeutical potential of this cytokine cannot be exhausted completely.

Occasionally, the administration of a short IL2 domain interacting with the receptor already suffices to attain at least some of the cellular effects (for example an immunostimulating effect). A study could show that an IL2 peptide with the amino acid sequence 1-30 IL2, however only in distinctly higher concentrations, has similar effects (Eckenberg, R., Xu, D., Moreau, J., Bossus, M., Mazie, J., Tartar, A., Liu, X., Alzari, P., Bertoglio, J., Thèze, J. (1997). Analysis of human IL-2/IL-” Receptor beta chain interactions: Monoclonal antibody H2-8 and new IL-2 Mutants define the critical role of alpha Helix-A of IL-z. Cytokine 9:488-498). This might be due to the fact that this peptide only binds to the β-chain of the receptor complex and with that only activates the medium-affine receptor complex. The like applies to other described sequences from another IL2 region (Eckenberg, R., Rose, T., Moreau, J.-L., Weil, R., Gesbert, F., Dubois, S., Tello, D., Bossus, M., Gras, H., Tartar, A., Bertoglio, J., Chouaib, S., Goldberg, M., Jacques, Y., Alzari, P M., Thèze, J. (2000) The first “-Helix of Interleukin(IL)-2 Folds as a Homotetramer, Acts as an Agonist of the IL-2 Receptor β Chain, and Induces Lymphokine-activated Killer Cells. J. Exp. Med. 3:529-39; Berndt, W., Chang, D., Smith, K., Ciardelli, T. (1994) Mutagenic analysis of a receptor contact site an interleukin 2: Preparation of an Interleukin-2 analog with increased potency. Biochemistry 33:6571-6577). However, all these peptides were synthesized without the purposive attachment on a support structure characterizing the present invention. Hence, they cannot contribute to the physiological receptor dimerization and exclusively function by medium- to low-affine receptor subunits. Thus, the activity thereof is weak. Moreover, these peptides have not been protected against proteolytic-degradation by incorporating protective groups. Hence, they suffer an accelerated in vivo degradation.

The present invention enables the provision of synthetic IL2R specific binding molecules with a high activity avoiding the drawbacks associated with the preparation and use of IL-2 or peptide fragments of IL-2.

Here, the side chain subunits preferably have at least one of the sequences

APTSSSTKKT₁ QLQLEHX¹X²X³X⁴ X⁵QMILNGINN
or
TIVX⁶FLNRWIT FX⁷QSX⁸ISTLT₁,

wherein

X¹ is selected from I or L,

X² is selected from I or L,

X³ is selected from V, L or M,

X⁴ is selected from E, D or K,

X⁵ is selected from L or F,

X⁶ is selected from E or D,

X⁷ is selected from A, G or C,

X⁸ is selected from A or I and

in the T₁ position a protective group according to the state of the art, preferably a carbohydrate moiety, preferably selected from glucose, mannose, N-acetyl glucosamine or N-acetylgalactosamine, is covalently bonded to threonine in a suitable manner as a protection against proteolytic degradation. According to the invention also binding molecules wherein the side chain subunits with the stated sequences have sequence homologies of at least 80%, preferably 90 or 95% may be used. In this case the sequence deviations especially are conservative amino acid exchanges. Preferred binding molecules have the structure:
APTSSSTKKT₁QLQLEHX¹X²X³X⁴X⁵QMILNGINN—Support Structure—TIVX⁶FLNRWIT FX⁷QSX⁸ISTLT₁;
TIVX⁶FLNRWITFX⁷QSX⁸ISTLT₁—Support Structure—APTSSSTKKT₁ QLQLEHX¹X²X³X⁴X⁵QMILNGINN;
APTSSSTKKT₁QLQLEHX¹X²X³X⁴X⁵QMILNGINN—Support Structure—APTSSSTKKT₁QLQLEHX⁰¹X⁰²X⁰³X⁰⁴X⁰⁵QMILNGINN
or TIVX⁶FLNRWIT FX⁷QSX⁸ISTLT₁—Support Structure—TIVX⁰⁶FLNRWIT FX⁰⁷QSX⁰⁸ISTLT₁

wherein the amino acids X⁰¹, X⁰², X⁰³, X⁰⁴ and X⁰⁵ are selected as indicated for X¹, X², X³, X⁴ and X⁵. According to the invention also binding molecules wherein the side chain subunits have a sequence homology to the stated sequences of at least 80%, preferably 90 or 95% may be used. In this case the sequence deviations especially are conservative amino acid exchanges.

The IL2R specific binding molecules can overcome the essential drawbacks of the state of the art. The binding molecules can be produced easily and economically. High production costs and the high physiological instability of recombinant molecules do not exist, the insufficient receptor stimulation observed with peptides does not occur since the binding molecules of the invention bind to the receptor complex in a functional cooperative manner in a defined spatial arrangement with the support structure having no affinity to the receptor. The receptor molecules of the invention also offer the possibility to increase the activity and to minimize side effects by a purposive sequence optimization.

The above-mentioned synthetic IL2R specific binding molecules are suited for the treatment of diseases of the immunologic system, e.g., inflammations and arthritic processes or of immunodeficiency syndromes of all types and genesis; diseases connected with an increased proliferation of cells, e.g., carcinoses, for example in the form of carcinomas, sarcomas, lymphomas and leukaemias; or infectious processes. It has already been known that recombinant interleukin 2 in combination with interleukin 12 has positive effects in the tumor therapy. In addition, such agents may also be combined with the pharmaceutical of the invention as preferred combination agents triggering apoptosis in target cells, in particular in tumor cells. In this case the expected positive effect on the therapeutic success is achieved by a local apoptosis within the tumor and a general immunostimulation by a pharmaceutical of the invention causing a local effectivity increase. Here, it is especially preferred to use TRAIL (TNF related apoptosis inducing ligand) receptor-specific binding and effector molecules.

While the abovementioned sequences and binding molecules are designed to act optimally on the human IL2-Receptor, it is easily possible to adapt the whole concept to the IL2-Receptor of any species, from which the homologous sequences are known.

The respective human sequences can be replaced by their animal counterparts and be combined in a way typical for this invention in order to optimize effects in an animal and thus to enable optimal veterinary use.

The Alignment given below shows the sequences relevant for cat and dog, which are homologous to the human sequences. These sequences are characterized for their ability to bind the respective receptors subunits in cat or dog. The invention includes all usual modifications and combinations of these side chains as outlined above for the human sequences. In preferred embodiments these sequences can be modified by introduction of conservative exchanges of amino acids in the sequences, by introduction of SH-containing residues and disulfide bridging as well as by other chemical modifications, which optimize the sterical design and stability of the resulting molecule. As Side chains they can be combined on a support structure according to the invention to become able to bind to receptor dimers.

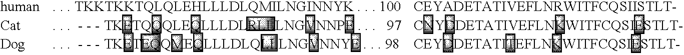

TRAIL Receptor-Specific Binding Molecules

TRAIL (TNF related apoptosis inducing ligand) is a protein belonging to the TNF family which selectively triggers the programmed cell death (apoptose) in tumor cells. The TRAIL specific binding and effector molecules synthesized here preferably on the basis of suitable cyclic support structures specifically bind to TRAIL receptors expressed by tumor cells and trigger apoptosis. Proteins triggering apoptosis such as the TNF (tumor necrosis factor) or Fas and AOP1 have been known for a longer time. Initially, the cytokine TNF was characterized as a protein having a strong tumoricide effect in mice. However, it could not therapeutically be used in humans due to its strong systemic toxicity. The same applies to Fas and APO1. TRAIL was described for the first time in 1999 (Walczak, H. et al. Nature Medicine 5 (1999) 157-163). This protein consists of 281 amino acids and is a type II membrane protein which is converted into a soluble molecule consisting of the amino acids 114-281 by cleavage. Subsequent to a spontaneous trimerization it binds to its specific receptors, thereby induces trimerization of the receptors. Thus it triggers the intracellular cell death program mediated by caspase. By now, four different receptors for TRAIL have been described: TRAIL-R1 and 2, both of which being involved in the signal transmission, and the receptors TRAIL R3 and 4, which, however, do not have a so-called “death domain” and hence cannot trigger signals after the binding of TRAIL. Both TRAIL itself and the mentioned receptors are expressed in most of the human tissues and organs; the selective tumoricide effect is probably based on the fact that normal cells have more of the “decoy receptors” 3 and 4 preventing an efficient signal triggering by inhibiting the trimerization of the active receptors.

In the amino acid sequence of naturally or recombinantly prepared TRAIL there are various domains which are responsible for the binding to the receptor and therefore for biological activity, namely the domains AA131-163, AA201-205, AA214-220, AA237-240, M258-283. These short domains positioned in sterical neighbourhood are cooperatively responsible for the interaction with the TRAIL receptor.

The present invention enables the provision of binding molecules specifically binding to TRAIL receptors with high affinity. In binding molecules of the invention the analogous domains mentioned in the above sections are coupled to cyclic support structures in a suitable cooperative arrangement in order to achieve the biological effect.

In preferred embodiments the TRAILR binding molecules of the invention exist in a trimerized form. Due to the covalent binding to a cyclic molecular subunit they have a high biological half life and due to the trimerization the biologically maximum possible activity with minimum side effects at the same time. These peptides are the minimum structure of RAIL required for the biological activity thereof. This achieves the highest specificity with the smallest side effects. In preferred embodiments the trimers may be prepared either homotrivalent or by a cyclic support structure or by a benzenetriamide linker.

Advantageously, the TRAIL peptides are prepared in a saccharide-modified form such that they have a high biological half life. This reduces the administration frequency and amount and with that the stress on the patient. By a purposive amino acid exchange the peptides can be converted on the one hand into a form being even more biologically active or a form having an increased biological half life or on the other hand into an antagonistic—i.e. depressant—form.

In one embodiment at least one side chain subunit of the TRAILR-specific binding molecule has the following sequence:

SKNEKALGRKINSWESSRSGHSFLS

In special embodiments the binding molecules with an affinity for TRAIL receptors have at least one side chain subunit with the sequence:

SKNEKALGRKIX₁X₂X₃X₄SSRX₅GHSFLS with

X₁=N or L,

X₂=S or Q or L or E,

X₃=W or L or R or A or Y,

X₄=E or N or A or D or H,

X₅=S or A.

Another subject of the invention are binding molecules wherein the side chain subunits have an sequence homology to the mentioned sequences of at least 80%, preferably 90 or 95% Here, the sequence deviations are in particular amino acid exchanges.

Special embodiments are those of the structural formulas

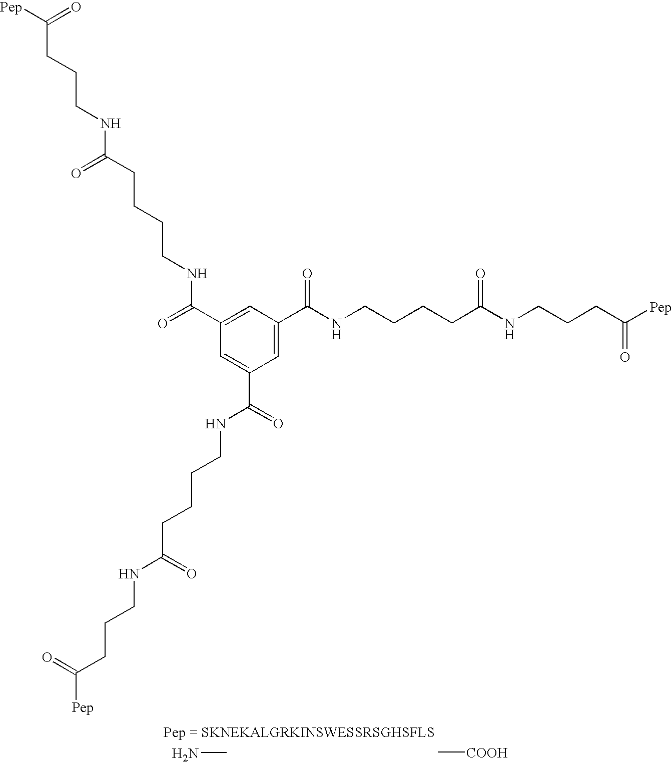

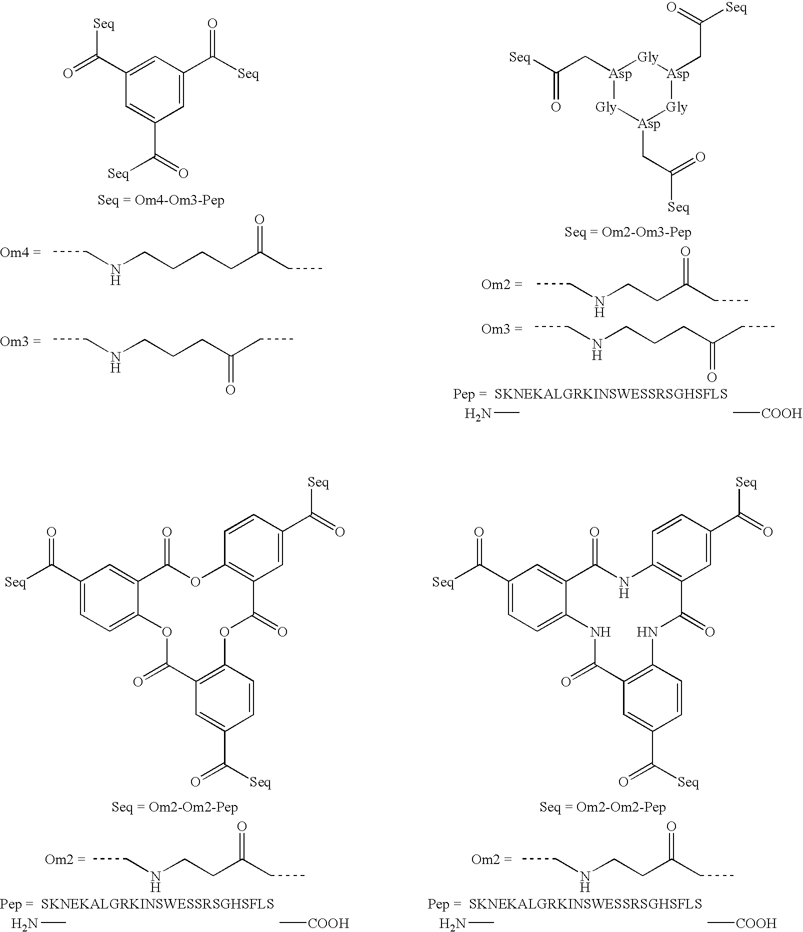

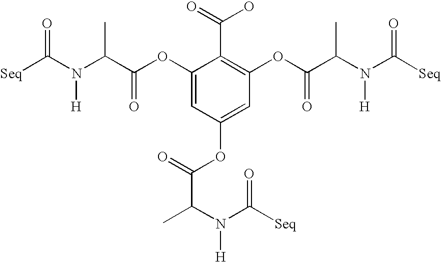

According to the invention, also binding molecules wherein the side chain subunits have a sequence homology to the mentioned sequences of at least 80%, preferably 90 or 95% are usable. Here, the sequence deviations are in particular conservative amino acid exchanges.

Antibodies and the Anti-Idiotypes Thereof

Another important, biologically active class of substances relevant for the present invention are antibodies. Antibodies are substances produced within the body which show a specific binding to substances which are usually foreign to the body and designated as antigens. Antibodies are of importance in the defence against pathogens and for the protection of the organism against infections. In various therapeutic and diagnostic applications antibodies having a relevant binding capacity for an planned indication are used or developed. Thus, antibodies are used to specifically enrich cytostatic agents or other tumor-impairing agents in tumors, or they are used to depict certain parts of the body or diseased area in imaging processes by loading them with a contrast agent. Antibodies may be isolated from the human body as natural antibodies. Normally, so-called monoclonal antibodies are antibodies which are grown in laboratory gnawers by immunization and subsequently immortalized by fusion of antibody-producing cells with myeloma cells and monocloned by isolation. Moreover, antibodies can also be monocloned by recombinant techniques transferring the coding genetic information in suitable, often bacterial or yeast-based expression systems. Recombinantly prepared antibodies may be obtained from all species with an immunoglobulin gene repertoire which is sufficiently sequenced and known. Hereinafter, according to the above description naturally occurring, monoclonally or recombinantly prepared antibodies will be referred to.

Antibodies are relatively large molecules, the specific binding area of which normally contains 6 hypervariable regions (based on the amino acid sequence). The binding properties of an antibody are defined by these so-called CDR (“Complementary Determining Regions”). However, in many cases not all of the 6 regions cooperatively participate in the binding and individual or several regions are not required for the specific binding in a critical manner. Both monoclonal antibodies and recombinantly prepared antibodies are therapeutically or diagnostically used in various indications. However, several drawbacks ensue in the therapeutical application of antibodies. On the one hand, the preparation of antibodies by both methods (monoclonally and recombinantly) is complicated and very expensive. On the other hand, antibodies themselves are natural molecules the patient's immunologic system reacts to. The therapy can substantially be impeded by the production of the patient's own antibodies (against the therapeutic antibodies) or by anaphylactic reactions. To overcome these problems, the methods for preparing therapeutic antibodies have to be changed and, e.g., mural monoclonal antibodies have to be introduced into the recombinant production in a molecular-biological, time-consuming and complicated process and at the same time humanized in this manner, i.e. optimized for the therapeutical use by incorporating human sequences.

According to the state of the art, already now it is possible to isolate anti-idiotypic substances from libraries, e.g., recombinant peptide libraries. Normally, the peptides isolated this way bind to the antibody only with low affinity and are not or only restrictedly able to competitively prevent the pathologic antibody formation.

The present invention solves the above-mentioned problems by providing synthetic binding molecules wherein the binding properties of a therapeutically relevant antibody are mimiced. The support structure of the invention enables to overcome the above-mentioned problems of the state of the art and to develop and design completely synthetic mimetics of the respective antibodies. The organic-chemical synthesis can be carried out more economical and with a better control than the recombinant preparation of an analogous molecule. Moreover, basically also unnatural amino acids or other suitable monomeric units of which a better compatibility, life time or immunotolerance is to be expected can be used.

However, antibodies may be important not only in therapy but they may also be pathogenetic reagents. Thus, antibodies are pathologic if they attack autologous tissue and thereby trigger a so-called autoimmune disease or if they are at least associated therewith. Another variant of the pathologic role of antibodies or other substances of the immunoglobuline superfamily exists in the challenge by allergenic substances in patients being allergic to the respective substance. Also these diseases are false reactions of the immunological apparatus, and the triggering thereof is initiated by the binding of the respective allergen. In all of these cases it would be desirable to neutralize the pathogenically relevant antibodies by reacting them with so-called anti-idiotypic substances. By anti-idiotypic it has to be understood that the substance specifically binds to the antibody's binding cavity just like the actual antigen. This prevents the pathologic binding to autologous antigens.

Another subject of the invention is the process for preparing a binding molecule, said process being performed as a solid phase synthesis process wherein the peptide of the side chain subunit bonded to the solid phase is successively extended by the respective amino acids and optionally a coupling to the support structure follows in a last step.

Another subject of the invention are peptides of the sequence

APTSSSTKKT₁ QLQLEHX¹X²X³X⁴ X⁵QMILNGINN
or
TIVX⁶FLNRWIT FX⁷QSX⁸ISTLT₁,
wherein

X¹ is selected from I or L,

X² is selected from I or L,

X³ is selected from V, L or M,

X⁴ is selected from E, D or K,

X⁵ is selected from L or F,

X⁶ is selected from E or D,

X⁷ is selected from A, G or C,

X⁸ is selected from A or I.

Another object of the invention are peptides wherein the side chain subunits have a sequence homology to the stated sequences of at least 80%, preferably 90 or 95% and which are not completely homologous to segments of the TRAIL sequence.

Another subject of the invention are pharmaceuticals and diagnostic agents containing the binding molecules or peptides of the invention.

The binding molecules of the invention are preferably used in the preparation of a pharmaceutical or diagnostic agent for the treatment or identification of diseases of the immunologic system, in particular inflammations, arthritic processes, immunodeficiency syndromes, auto-immune syndromes and immunodeficiency syndromes, fertility disturbances, for the contraception or antiviral prophylaxis, diseases connected with an increased proliferation of cells, in particular tumor diseases, carcinoses, sarcomas, lymphomas and leukaemias; and/or infectious processes with humans or animals.

The pharmaceutical of the invention is provided in particular in the form of a micro encapsulation, liposomal preparation or depot preparation containing suitable additives, carriers, adjuvants and/or at least one additional active substance. Optionally, the pharmaceutical of the invention is provided in combination with a common suitable pharmaceutical carrier. For example, suitable carriers include buffered sodium chloride solutions, water, emulsions, e.g., oil/water emulsions, wetting agents, sterile solutions, etc. The pharmaceutical of the invention may be provided in the form of an injection solution, a tablet, an oinment, a suspension, an emulsion, a suppository, an aerosol, etc. administered in the form of depots (microcapsules, zinc salts, liposome, etc.). The peptides may be microencapsulated due to the small size thereof resulting in depot forms with different durations of effect according to the respective pore size of the capsules. The depot form may be administered locally such that the highest concentration is ensured in the required area (e.g., initial carcinoma) over a long period of time. Inter alia, the mode of administration of the pharmaceutical depends on the form in which the active substance is present; it may be performed orally or parenterally. The skilled person knows several methods. The suitable dosage is determined by the attending physician and depends on several factors, e.g., the age, sex, weight of the patient, the nature and the stage of the disease, the mode of administration, etc.

In a preferred embodiment the use of a binding molecule of the invention takes place in particular IL2-specifically by using said molecule for the preparation of human LAK (Lymphokine Activated Killer Cells) ex vivo. In such an ex vivo therapy, cells are taken from the patient, treated with the active substance and activated in a culture dish and subsequently re-infused into the patient.

In particular embodiments of the invention interleukin 12, an interleukin 12 receptor-specific binding and effector molecule, or recombinant IL12 is used as an additional active substance.

In another embodiment TRAIL (TNF related apoptosis inducing ligand), a TRAIL receptor-specific binding and effector molecule, or recombinantly prepared TRAIL is used as an additional active substance.

In a special embodiment a virostatic, in particular a virostatic capable of retarding or blocking the entry of virus particles, in particular HIV particles, into target cells, in particular T-lymphocytes, is added to the pharmaceutical of the invention as an additional active substance.

Another subject of the invention is a binding molecule wherein the support structure consists of a linear peptide of 1-10 amino acids selected from G, beta-D,L-alanine or NH₂(CH₂)ₓCOOH (with x=3-8).

### EXAMPLE 1

Multimerization Principle of Peptides on the Basis of Cyclopeptides

A: Antiparallel Dimers:

A cyclic peptide is generated and used as the starting point for the solid phase synthesis. Initially, the first peptide strand is synthesized on the solid phase, then the cyclopeptide is coupled thereto and subsequently the second peptide strand is either synthesized on this loaded resin step by step or the previously purified peptide B is coupled thereto. 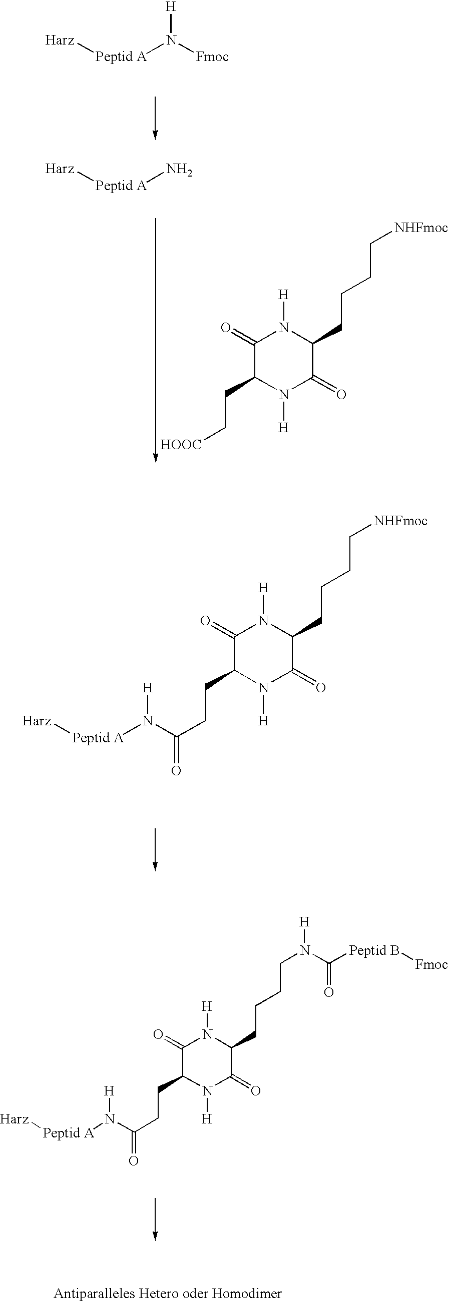
Coupling of Lysine Cyclopeptides and Asparaginic Acid in Solution

Zero point two ml of NMM and 1 mmol of CDMT are added to a solution of 1 mmol of the asparagines acid derivative in 10 ml of methylene chloride cooled to 0° C. under an argon atmosphere, and this mixture is stirred at 0° C. for 2 h. Subsequently, additional 0.2 ml of NMM and 1 mole equivalent of the cyclopeptide in 10 ml of absolute DMF are added. This mixture is stirred at 0° C. for 2 h and at room temperature over night, 50 ml of ice water are added, and it is extracted twice with 100 ml of ethyl acetate each. The combined organic phases are extracted by shaking with saturated sodium hydrogencarbonate solution and 5% of sodium hydrogensulfate solution. After drying over sodium sulfate, the remaining solution is concentrated in a Rotavapor and the residue is suspended in methylene chloride, where the product is obtained in the form of a white solid.

| Batch size | | | |
|---|---|---|---|
| Boc-L-Asp(OBz) | 162 | mg | 0.5 mmol |
| cyclo-L-Ala-L-Lys | 100 | mg | 0.5 mmol |
| CDMT | 89 | mg | |
| NMM | 0.2 | ml | |
| Methylene chloride | 5 | ml | |
| DMF | 5 | ml | |
| Yield: | | | |
| Theoretical: | 0.25 | g | 100% |
| Actual: | 0.18 | g | 72% |

B: Parallel Dimers

A cyclohexapeptide of the sequence D-K-D-K-D-K is generated according to the scheme mentioned below. By selecting the protective group appropriately, the cyclopeptide can be bonded to a polymer support by the side chain of one of the asparaginic acids, optionally by spacer molecules, and then three peptide strands can be synthesized on the resin in parallel or the purified peptides can be coupled to a solid phase.

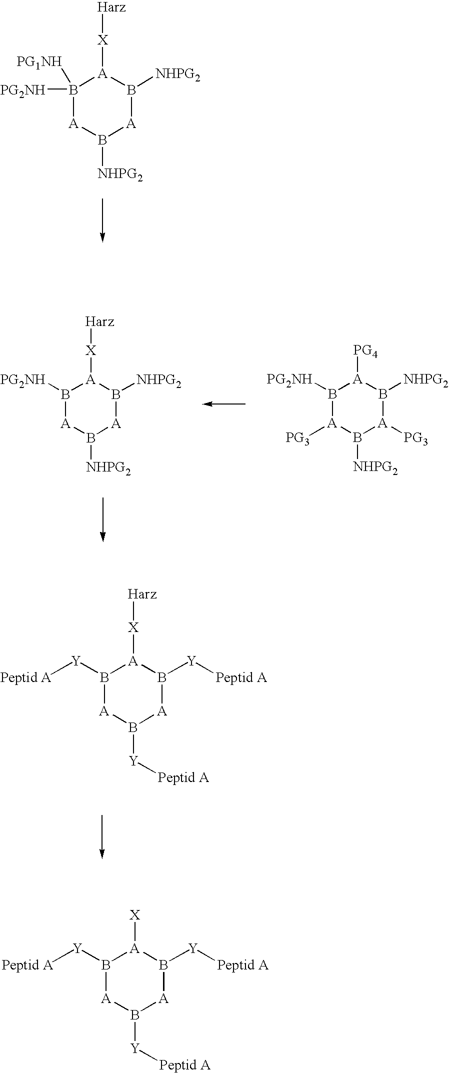

A=DB=KX, Y=spacer molecules

PG1/PG2=orthogonal protective groups

PG3/PG4=orthogonal protective groups

### EXAMPLE 2

Embodiment for peptide A according to example 1B in the form of IL2R-specific binding molecules:

The peptides are synthesized as follows:

Synthesis: Using solid phase synthesis (100 mg Wang resin (0.4 mmol/g) per assay), peptides having variable sequences are automatically prepared in a synthesis robot (Sophas-3, Zinsser Analytik). As monomer units Fmoc-derivatized amino acids (c=0.5 mol/l) with suitable additional protective groups according to the state of the art were used. The amino acid units and coupling reagents (HOBt, c=0.766 g/ml; PyBOP, c=0.2602 g/ml) were provided dissolved in DMF. The individual coupling steps were performed for 2×45 min each and the resin was washed with DMF between the steps. The cleavage of Fmoc was performed by a 20 min treatment with 50% (v/v) of piperidine in DMF. The subsequent cleavage from the resin was performed in the presence of a mixture of 95% of trifluoroacetic acid, 2.5% of water, 2.5% of diisopropylsilane for 20 min. The cleaved peptides were precipitated by adding tert-butyl methyl ether, centrifuged off and again combined with tert-butyl methyl ether and centrifuged off. Subsequently, the peptides were lyophilized and purified by reversed phase HPLC and characterized by LC/MS (Thermoquest LCQDuo). The purified peptides were checked in a cell culture for the cytotoxicity increasing effect thereof.

The peptides with the structures

| | |
|---|---|
| Sequence 1: | APTSSSTKKT QLQLEHLLLK LQMILNGINN |
| Sequence 2: | APTSSSTKKT QLQLEHFLLD FQMILNGINN |
| Sequence 3: | APTSSSTKKT QLQLEHFLMK FQMILNGINN |

were prepared and tested.

Cytotoxicity Test:

For the cytotoxicity measurement, NK cells, which specifically kill tumor cells, as effector cells were incubated with tumor cells as target cells in the presence of the test peptides. The cytotoxicity measurement was performed by measuring the release of lactate dehydrogenase (LDH) according to the standard protocol by a cytotoxicity test kit (Promega, Germany) and in a microwell plate reader. As NK cells (E), YT cells (DSMZ, Germany) were used. As target cells (T), Daudi cells (DSMZ, Germany) were used. YT and Daudi cells were seeded in microwell plates at a ratio of 1:10 and incubated in the presence of 0.3, 3, 30:M test peptide for 16 h. Human IL-2 (Sigma) in a concentration of 5 nM served as the positive control.

On the next day, 10:l of lysis buffer each were added to the cells and the cells were incubated for additional 2 h. Subsequently, 5:l of the agent were transferred into a second microwell plate and combined with 50:l of substrate buffer. After a 30 min incubation, the reaction was terminated by 50:l of stop solution and the absorption of the formed MTT color complex was measured in a microwell plate reader.

Table 1 illustrates that already in a concentration of 0.3:M the cytotoxicity inducing effect of the described test peptides is equally high as that of human IL-2.

TABLE 1
Overview of Results for the Cytotoxicity Test

| Substance (sequence #) | Cytotoxicity |
|---|---|
| Control | 10% |
| IL-2 | 60% |
| Sequence 1 30:M | 30% |
| Sequence 1 3:M | 30% |
| Sequence 1 0.3:M | 30% |
| Sequence 2 30:M | 10% |
| Sequence 2 3:M | 80% |
| Sequence 2 0.3:M | 30% |
| Sequence 3 30:M | 0% |
| Sequence 3 3:M | 20% |
| Sequence 3 0.3:M | 0% |

### EXAMPLE 3

Synthesis of the Trihydroxybenzoic Acid Support Structure

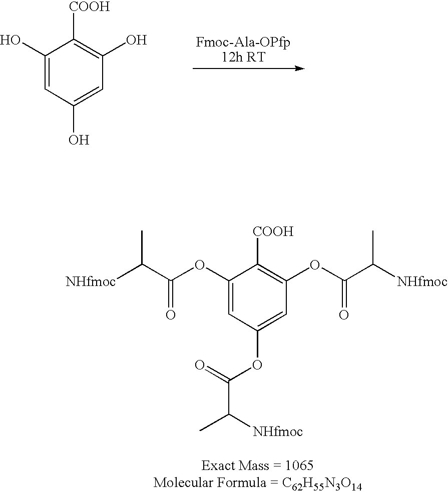

Assay:

| | | | | | | |
|---|---|---|---|---|---|---|
| 2,4,6-trihydroxybenzoic Acid | 170 | mol | 4 | mol | 0.68 | g |
| Fmoc-Ala-OPfp | 477.4 | g/mol | 16 | mmol | 7.16 | g |
| HOBt | 135.1 | g/mol | 13 | mmol | 1.76 | g |
| DMF | | | | | 100 | ml |

Four mmol trihydroxybenzoic acid are dissolved in as little aqueous sodium carbonate solution (pH=9) as possible and added to a suspension of 16 mmol of the pentafluorophenol ester in 130 ml of acetone. The mixture is stirred at room temperature overnight and acidified with 5% of HCl at 0° C. to pH=1. The solution is concentrated in the Rotavapor to half of the initial volume and extracted by shaking with 2×150 ml of ethyl acetate. The combined organic phases are extracted by shaking with 100 ml of water and 5× with concentrated sodium hydrogensulfate solution and dried over sodium sulfate. After removing the volatile components in the Rotavapor, the residue is suspended in methanol, filtered off under suction and rewashed with cold methanol. If the product does not precipitate, it is completely rotary-evaporated.

### EXAMPLE 3

Synthesis of an Interleukin-2 Immunomer

Chemical Formula of the substance to be synthesized:

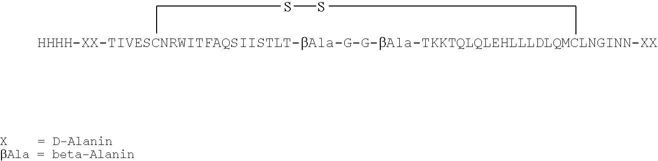

| Abbreviations | Substance |
|---|---|
| DMF | N,N-Dimethylformamide (peptide synthesis grade) |
| DCM | Dichloromethane (peptide synthesis grade) |
| HOBT | 1-Hydroxybenzotriazole (anhydrous) |
| DBU | 1,8-Diazabicyclo[5,4,0]undec-7-en 98% |
| PyBOP | Benzotriazole-1-yl-oxy-tris-pyrrolidino-phosphonium- |
| | hexafluorophosphate |
| DIPEA | N-Ethyldiisopropylamin 98+% |
| TIS | Triisopropylsilan 99% |
| MeOH | Methanol HPLC (gradient grade) |
| TFE | 2,2,2-Trifluorethanol 99.8% |
| EDT | Ethanedithiol |

General Strategy

The synthesis is carried out on 2-chlorotrityl chloride resin (200-400 mesh) with a substitution rate of 0.2 mmol/g. The first seven amino acids are coupled as a fragment. Attachement of the following amino acids is achieved by single, double or triple coupling with 10 fold excess of amino acids and PyBOP/HOBT/DIPEA as coupling additives. N-terminal deprotection of the growing peptide chain is achieved by double treatment with piperidine/DMF (1/3). In difficult cases a third treatment is done with DBU/piperidine/DMF (2/2/1996). The number of coupling and deprotection cycles used and is shown in the following scheme (Information on difficult couplings/deprotections gained by monitoring of each elongation by HPLC-MS.)

RES-XXNNIGN-LCMQLDLLLHELQLQTKKTBGGB-TLTSIISQAFTIWR
NCSEVITXXHHHH

| | |
|---|---|
| COUPLING: | 22221211111111122222222-223333333333333 |
| | 333333333333 |
| DEPROTECT: | 22222222222222322222222-223333333333333 |
| | 333333333333 |

1,2,3 = number of coupling or deprotection cycles
X = D-Alanine
B = β-Alanine

Protocol 1: Synthesis of the First Peptide Fragment

2 mmol of the first amino acid and 4 mmol DIPEA are dissolved in 10 ml dry DCM. This solution is added to 1.0 g of dry 2-chlorotrityl chloride resin (200-400 mesh) and the mixture allowed to react for 60 minutes on a vortexer. At the end of the reaction the resin is allowed to react twice with 20 ml of DCM/MeOH/DIPEA for 3 minutes, washed twice with 20 ml DCM and twice with DMF. The resin is then treated twice with 20 ml piperidine/DMF (1:3) for 3/20 minutes and washed 6 times with 20 ml DMF. The attachment of amino acids 2-7 is achieved by the following procedure: 5 mmol of the amino acid, 7.5 mmol HOBT and 10 mmol DIPEA are dissolved in 15 ml DMF. After 5 minutes 5 mmol of PyBOP and 10 mmol DIPEA are added and this solution is poured onto the resin. After 60 minutes on a vortexer the resin is washed 6 times with 20 ml DMF, treated twice with 20 ml piperidine/DMF (1:3) for 20/20 minutes and washed 6 times with 20 ml DMF. In case of the N-terminal amino acid the resin is not treated with piperidine/DMF.

Protocol 2: Cleavage of the Peptide Fragment

After attachement of the last amino acid the resin is washed 6 times with 20 ml DMF, twice with 20 ml DCM and then allowed to react with 50 ml TFE/DCM (2/8) for 60 minutes. The resin is filtered off, the solvents removed in vacuo and the crude peptide fragment used without further purification.

Protocol 3: Reattachement of the Peptide Fragment

1 mmol of the fragment and 2 mmol DIPEA are dissolved in 50 ml dry DCM. This solution is added to 5.0 g of dry 2-chlorotrityl chloride resin (200-400 mesh) and the mixture allowed to react for 12 hours on a vortexer. At the end of the reaction the resin is allowed to react twice with 50 ml of DCM/MeOH/DIPEA for 3 minutes, washed twice with 50 ml DCM and twice with 50 ml DMF.

Protocol 4: Coupling of Amino Acids 8-57

The dried resin is allowed to swell in 50 ml piperidine/DMF (1/3) for 30 minutes, treated with 30 ml piperidine/DMF (1/3) for 20 minutes, treated with 30 ml of DBU/piperidine/DMF (2/2/96) for 20 minutes and washed 6 times with 30 ml DMF. 10 mmol of the amino acid, 15 mmol HOBT and 20 mmol DIPEA are dissolved in 30 ml DMF. After 5 minutes 10 mmol of PyBOP and 20 mmol DIPEA are added and this solution is poured onto the resin. After 60 minutes on a vortexer the resin is washed twice with 30 ml DMF and the coupling repeated once or (in difficult cases) twice for 60 minutes. The resin is then washed 6 times with 30 ml DMF. The resin can directly be used to couple the next amino acid or—after 2 washings with 30 ml DCM—dried in vacuum and stored at −80° C.

Protocol 5: Cleavage and Deprotection

After coupling of the last amino acid the N-terminal protecting group is removed by a double treatment with 30 ml piperidine/DMF (1/3) for 20 minutes and treatment with DBU/piperidine/DMF (2/2/1996) for 20 minutes. The resin is washed 6 times with 30 ml DMF, twice with 30 ml DCM and treated with 50 ml TFE/DCM (2/8) for 180 minutes. After filtration the solvent is removed in vacuo and the protecting groups removed by treatment with 30 ml TFA/TIS/EDT/water (94/1/2.5/2.5) for 180 minutes under an inert atmosphere. This solution is poured into 300 ml cold ether, the precipitate dissolved in methanol and the peptide purified by RP-HPLC (Kromasil 100 C4 10 μm, 250×4.6 mm) and the collected fractions directly used for the refolding procedure.

Protocol 6: Refolding Procedure

The respective elution fractions were diluted to a final volume of 20 ml using exactly the same solvent as being present in the elution fraction. This solution of the purified product was incubated for 30 min. at room temperature with 10 ml Ni-NTA Superflow (Qiagen) under slight agitation in a beaker. The Superflow particles were packed into an empty FPLC Column and attached to an FPLC-machine. Using the chromatography programme of this machine, the solvent was exchanged in a 10 min. Gradient by Water, then another 10 min. gradient was used to change the solvent to a mixture of water/trifluorethanol (1/1). During a 24 h period, this solvent was oxygenized by bubbling oxygen through the reservoir bottle and using oxygen as atmosphere in the bottle in order to close the disulfide bridge. During oxygenation, a constant flow of 1 ml/min was passed for 24 h along the column, while the eluate was constantly recirculated into the reservoir bottle.

At the end of the 24 h period, the final product, being refolded and sealed by disulfide bridging was eluted by the use of Phosphate Buffered Saline (PBS, pH 7,2) containing 150 mM imidazole or by using 0.1M Acetate buffer (pH 4.5).

The binding molecule is able to bind to the beta/gamma Interleukin 2 Receptor Heterodimer, to activate it and to induce signal transduction.

1: A binding molecule comprising a support structure of at least one cyclic molecular subunit and at least two side chain subunits, characterized in that the side chain subunits are polypeptide chains consisting of natural and/or unnatural D- and/or L-amino acids and/or polynucleotide chains and the side chain subunits are covalently bonded to the support structure.

2: The binding molecule of claim 1, characterized in that the cyclic molecular subunit is a cyclic polypeptide synthesized from natural or unnatural D- and/or L-amino acids, an aromatic ring, an aromatic ring system, a polylactone, a polylactam, benzenetriamide, a trilactone or a trilactam.

3: The binding molecule of claim 2, characterized in that the amino acids forming the cyclic polypeptide are linked by polypeptide bonds.

4: The binding molecule of claim 2, characterized in that the cyclic molecular subunit is a cyclic polypeptide and said at least two side chain subunits are linked to the cyclic molecular subunit by amino acid side chain moieties of the amino acids lysine, serine, threonine, glutamate, asparagine and/or aspartate of the cyclic molecular subunit.

5: The binding molecule of claim 2 characterized in that the cyclic polypeptide consists of 2 to 10, preferably 2, 3, 4 or 6 amino acids.

6: The binding molecule of claim 1, characterized in that has one of the structures:
a) benzenetriamide: 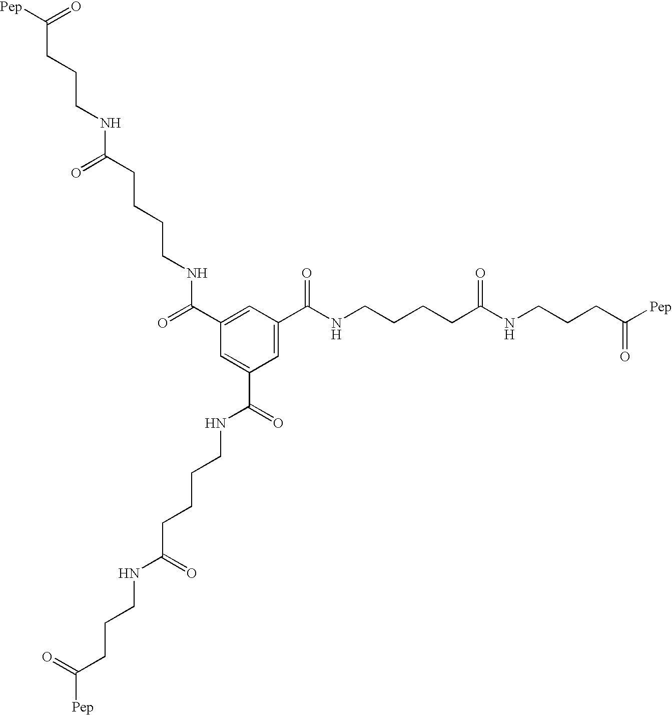
b) cyclohexapeptide: 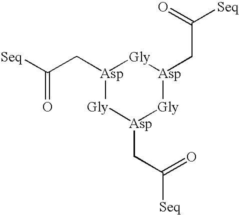
or a cyclohexapeptide of the sequence DKDKDK with one up to three side chain peptides Seq,
c) trilactone: 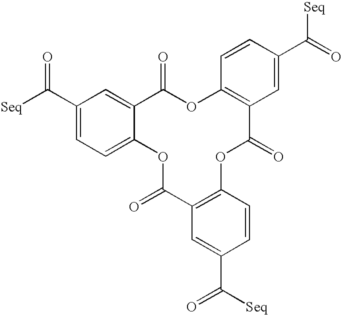
d) trilactame: 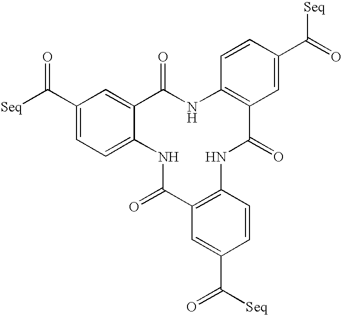
e) trihydroxybenzoic acid support structure: 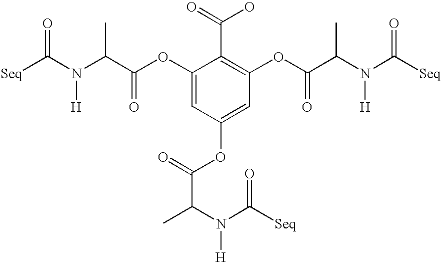
f) cyclic dipeptide: cyclo-L-Ala-L-Lys, derivatized with Seq, wherein Seq or Pep represent the side chain subunits or —OH.

7: The binding molecule of claim 1, characterized in that the polypeptide chains have the same amino acid sequence.

8: The binding molecule of claim 1, characterized in that the polypeptide chains consist of 5 to 60, preferably 10 to 50, in particular 12 to 40 amino acids.

9: The binding molecule of claim 1, characterized in that the side chain subunits are bonded to the support structure by spacer molecular subunits (spacers) especially consisting of carbohydrates or nucleic acids.

10: The binding molecule of claim 1, characterized in that the unnatural amino acids have the formula NH₂(CH₂)ₙCOOH with n=2 to 8, in particular NH₂(CH₂)₂COOH, NH₂(CH₂)₃COOH or NH₂(CH₂)₄COOH.

11: The binding molecule of claim 1, wherein the side chain subunits attached to the support structure have at least one of the sequences

| | |
|---|---|
| APTSSSTKKT₁ QLQLEHX¹X²X³X⁴ X⁵QMILNGINN | (SC1) |
| or | |
| TIVX⁶FLNRWIT FX⁷QSX⁸ISTLT₁, | (SC2) |
| or | |
| TKETQQQLEQLLLDLRLLLNGVNNPE | (SC3) |
| or | |
| TKETEQQMEQLLLDLQLLLNGVNNYE | (SC4) |
| or | |
| NYDDETATIVEFLNKWITFAQSIFSTLT | (SC5) |
| or | |
| EYDDETATITEFLNKWITFAQSIFSTLT, | (SC6) |

wherein
X¹ is selected from I or L,
X² is selected from I or L,
X³ is selected from V, L or M,
X⁴ is selected from E, D or K,
X⁵ is selected from L or F,
X⁶ is selected from E or D,
X⁷ is selected from A, G or C,
X⁸ is selected from A or I;
at the T₁ threonine a protective group according to the state of the art, preferably a carbohydrate moiety, preferably selected from glucose, mannose, N-acetyl glucosamine or N-acetylgalactosamine, is optionally covalently bonded in a suitable manner as a protection against proteolytic degradation;
protection against exopeptidases is optionally achieved by attachment of two D-amino acid residues at the free C- and/or N-Terminus;
the sequences are optionally modified by replacement of single residues by residues with SH-group(s) in order to prepare for disulfide bridging between the polypeptide side chains after attachment to the support structure;
and binding molecules, wherein the side chain subunits have sequence homologies to the stated sequences of at least 80%;
and fragments of the above mentioned sequences, which are shortened for maximally 6 amino acids from the N- or the C-terminus.

12: A binding molecule of claim 11 having the structure
SCX—Support Structure—SCY
wherein SCX (Side Chain X) can be any of the six side Chains (SC1-6) of claim 11, and SCY (Side Chain Y) can be any of the side chains (SC1-6).

13: The binding molecule of claim 1, wherein at least one side chain subunit has the sequence
SKNEKALGRKIX₁X₂X₃X₄SSRX₅GHSFLS,
with
X₁=N or L,
X₂=S or Q or L or E,
X₃=W or L or R or A or Y,
X₄=E or N or A or D or H,
X₅=S or A,
and binding molecules wherein the side chain subunits have sequence homologies to the stated sequences of at least 80%.

14: The binding molecule of claim 1, characterized in that it has one of the structural formulas: 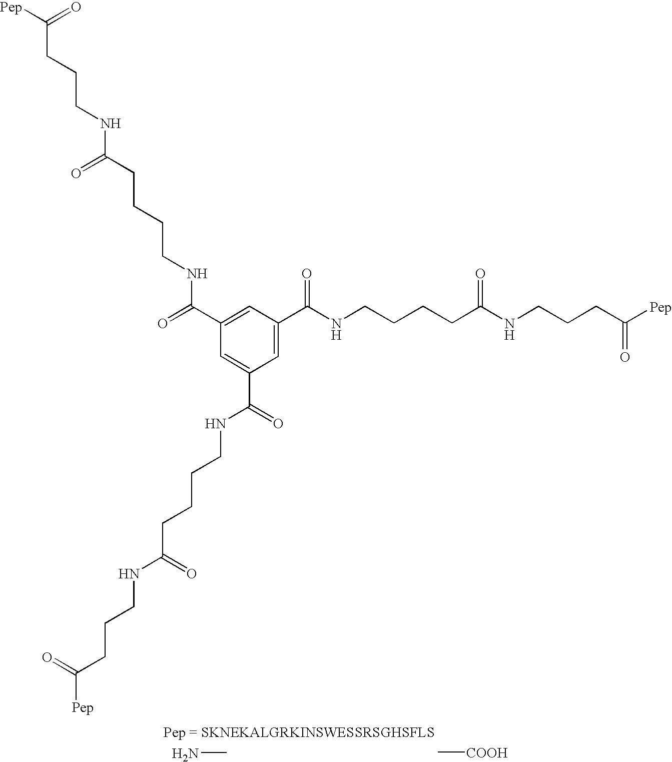 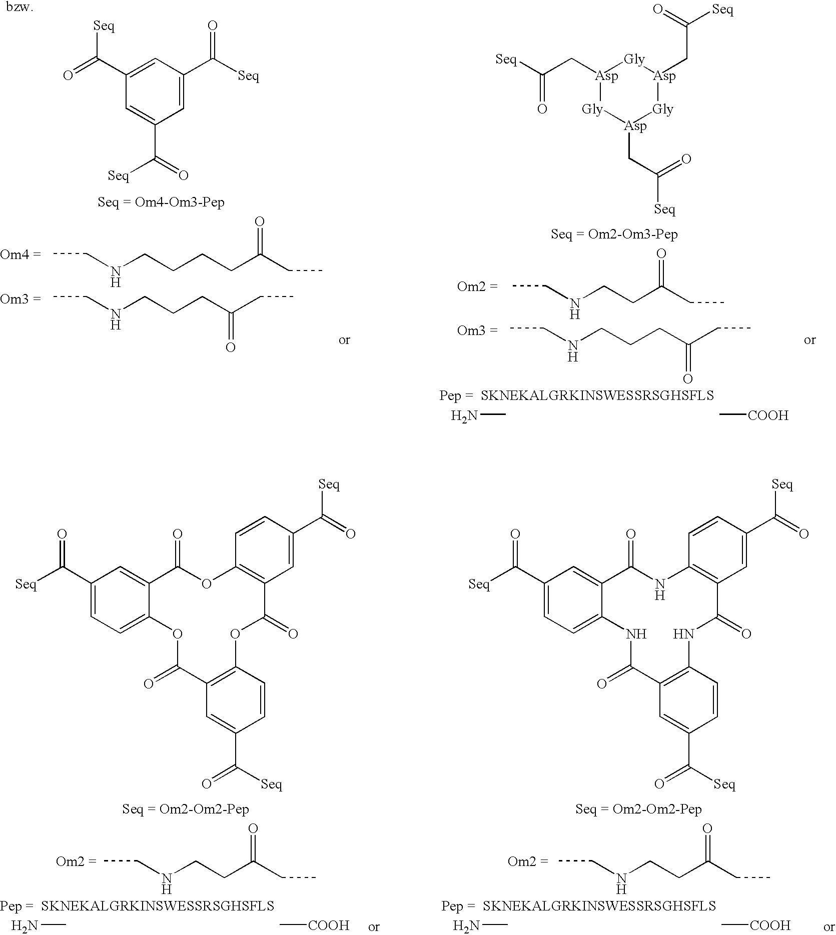 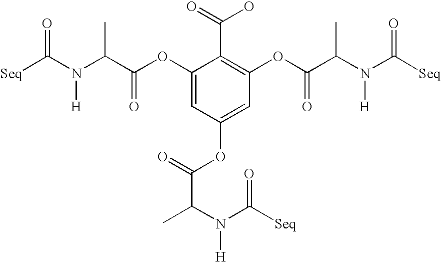
and binding molecules wherein the side chain subunits have sequence homologies to the stated sequences of at least 80%.

15: A process for the preparation of a binding molecule according to claim 1, wherein the process is performed as a solid phase synthesis process wherein the peptide of the side chain subunit is bonded to the solid phase, successively extended by the respective amino acids and optionally a coupling to the support structure follows in a last step.

16: A peptide having the structure
APTSSSTKKT₁ QLQLEHX¹X²X³X⁴X⁵QMILNGINN
or
TIVX⁶FLNRWIT FX⁷QSX⁸ISTLT₁,
wherein
X¹ is selected from I or L,
X² is selected from I or L,
X³ is selected from V, L or M,
X⁴ is selected from E, D or K,
X⁵ is selected from L or F,
X⁶ is selected from E or D,
X⁷ is selected from A, G or C,
X⁸ is selected from A or I,
and binding molecules wherein the side chain subunits have a sequence homology to the stated sequences of at least 80%.

17: A pharmaceutical containing binding molecules comprising a support structure of at least one cyclic molecular subunit and at least two side chain subunits, characterized in that the side chain subunits are polypeptide chains consisting of natural and/or unnatural D- and/or L-amino acids and/or polynucleotide chains and the side chain subunits are covalently bonded to the support structure and/or peptides according to claim 16.

18: Diagnostic agent containing binding molecules comprising a support structure of at least one cyclic molecular subunit and at least two side chain subunits, characterized in that the side chain subunits are polypeptide chains consisting of natural and/or unnatural D- and/or L-amino acids and/or polynucleotide chains and the side chain subunits are covalently bonded to the support structure and/or peptides according to claim 16.

19: Use of binding molecules comprising a support structure of at least one cyclic molecular subunit and at least two side chain subunits, characterized in that the side chain subunits are polypeptide chains consisting of natural and/or unnatural D- and/or L-amino acids and/or polynucleotide chains and the side chain subunits are covalently bonded to the support structure and/or peptides according to claim 16 for preparing a pharmaceutical or diagnostic agent for the treatment or detection of diseases of the immunologic system, in particular inflammations, arthritic processes, immunodeficiency syndromes, auto-immune syndromes and immunodeficiency syndromes, fertility disturbances, for the contraception or antiviral prophylaxis, diseases connected with an increased proliferation of cells, in particular tumor diseases, carcinoses, sarcomas, lymphomas and leukaemias; and/or infectious processes with humans or animals.

20: The pharmaceutical according to claim 17 in the form of a microencapsulation, liposomal preparation or depot preparation containing suitable additives, carriers, adjuvants and/or at least one additional active substance.

21: The pharmaceutical according to claim 17, characterized in that interleukin 12, an interleukin 12 receptor-specific binding and effector molecule or recombinant IL12 is used as the additional active substance.

22: The pharmaceutical according to claim 17, characterized in that TRAIL (TNF related apoptosis inducing ligand), a TRAIL receptor specific binding and effector molecule or recombinantly prepared TRAIL is used as the additional active substance.

23: The pharmaceutical according to claim 17, characterized in that a virostatic, in particular a virostatic capable of retarding or blocking the entry of virus particles, in particular HIV particles, into target cells, in particular T-lymphocytes, is used as the additional active substance.

24: The binding molecule according to claim 1, characterized in that exactly two side chains are bonded to the support structure and that the support structure is minimized to a linear peptide of 2-10 amino acids comprising at least two different amino acids selected from G, P, beta-alanine or NH₂(CH₂)ₓCOOH (with x=3-8).

25: The binding molecule of claim 24, defined by the formula [chemical structure]
